(19) United States
(12) Patent Application Publication
(10) Pub. No.: US 2019/0314163 A1
(43) Pub. Date: Oct. 17, 2019

(54) SPINAL IMPLANT AND RELATED METHODS

(71) Applicant: Brahm Holdings, LLC, Germantown, TN (US)

(72) Inventor: Timothy R. BRAHM, Germantown, TN (US)

(21) Appl. No.: 16/385,230

(22) Filed: Apr. 16, 2019

Related U.S. Application Data
(60) Provisional application No. 62/658,162, filed on Apr. 16, 2018.

(51) Int. Cl. A61F 2/44; A61F 2/46; A61L 27/36

(52) U.S. Cl. CPC A61F 2/442; A61F 2/4611; A61F 2/4601; A61F 2/4455; A61F 2002/30059; A61L 27/365; A61F 2220/0025; A61L 2430/40; A61L 27/3608

(57) ABSTRACT

A spinal implant that functions as a vector for delivering a flowable biologic is provided. Methods for stabilizing and treating the cervical spine are also provided.

### CROSS-REFERENCE TO RELATED APPLICATIONS

The present application claims priority to U.S. Ser. No. 62/658,162 filed Apr. 16, 2018, the contents of which are incorporated in their entirety.

### FIELD OF THE INVENTION

An compressible and expandable cervical implant is provided to give spinal implant components the ability to act as a vector. Methods for stabilizing and treating the spine following such surgery are also provided.

### BACKGROUND OF THE INVENTION

Cervical radiculopathy occurs when a cervical spinal nerve root is compressed and causes radicular, or radiating, symptoms throughout the neck and upper extremities. The cervical spinal nerve root can become compressed due to degenerative changes to bone or tissue over time such as, for example, cervical degenerative disc disease, cervical herniated discs, cervical bulging discs, bone spurs and cervical spinal stenosis. A compressed nerve can cause distress signals along nerve branches causing pain, tingling, numbness and muscle weakness.

Treatment of cervical radiculopathy is usually carried out initially with a corticosteroid or non-steroidal pain medication. In the event such therapy is not successful, physical therapy is attempted followed by open spine surgery. Particularly, decompression and stabilization surgery may be carried out. One of a variety of decompression surgeries may be performed including laminectomy, laminotomy, foraminotomy, or laminaplasty. Spinal fusion, a surgical technique used to join two or more vertebrae, is another surgical intervention option that is often performed in combination with such decompression procedures to immobilize the affected vertebrae and stabilize sections of the spine. Fusion may use a combination of bone implant(s), rods and screws to connect to vertebrae together, thereby inducing bony incorporation and healing the vertebrae together as one piece of bone. Fusion helps prevent recurrence of spinal stenosis and aids in eliminating pain arising from an unstable spine. Supplementary bone tissue (e.g., autologous iliac crest bone, alloimplant tissue, synthetic cage with bone substitute filler) is used in conjunction with the body's natural bone growth processes to fuse the vertebrae. Autologous tissue recovery, however, often leads to implant site morbidity. Furthermore, fusion often leads to adjacent segment disease, which may limit the duration of success of the operation. Disc replacement may also be carried out. Such a surgery replaces a problematic disc with an artificial disc and maintains mobility at the particular point in the cervical spine.

Thus, there remains a need for safe, effective interbody spine implants that are both compressible and expandable as well as methods for stabilizing and treating the spinal region.

### SUMMARY OF THE INVENTION

According to one aspect, a spinal implant is provided. The spinal implant includes at least one structural material selected from the group consisting of metal, plastic, cancellous bone, demineralized cancellous bone, fresh alloimplant, frozen alloimplant freeze dried bone alloimplant, demineralized freeze dried bone alloimplant, cortical cancellous bone, or a combination thereof. The spinal implant further includes an effective amount of at least one flowable biologic component selected from the group consisting of Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a birth tissue composition, and any combination thereof. A effective amount of the flowable biologic component is deposited on, in, or around the structural material such that the structural material becomes a vector (wicks up) the flowable biologic component for delivery of the at least one flowable biologic component on, in or around a patient in need of treatment. According to one embodiment, the birth tissue material composition includes one or more components of the placental organ from a mammal, the one or more of the components of the placental organ selected from the group consisting of umbilical cord, umbilical cord blood, chorionic membrane, amnion membrane, amniotic membrane, Wharton's jelly, amniotic fluid, and extracellular material. According to one embodiment, the implant further includes one or more natural or recombinant bone morphogenetic proteins (BMPs) selected from the group consisting of BMP-2, BMP-7 and a combination thereof. According to one embodiment, one or more synthetic-based bone implant extenders. According to one embodiment, the one or more synthetic-based bone implant extenders is calcium phosphate, tricalcium phosphate, phosphate, calcium sulfate, bioactive glass, or a combination thereof. According to one embodiment, the implant further includes one or more bioactive agents. According to one embodiment, the implant further includes one or more synthetic polymers. According to one embodiment, the implant is shaped to fuse to one or more of the C1, C2, C3-C6 or C7 discs. According to one embodiment, the flowable biologic component further includes one or more vitamin, nutrient, inflammatory inhibitor, antibiotic, cytokine, mineral, growth factor hyaluronic acid, cellular attractant, scaffolding reagent, antibiotic, chemotherapeutic agent, antigen, antibody, enzyme, NSAID, or muscle relaxant.

According to one aspect, a method of stabilizing a vertebral region of a spine is provided. The method includes the step of providing an implant as provided herein. The method further includes the step of applying the implant to, into, or on a vertebral region of the spine. According to one embodiment, the method further includes the step of securing the implant to the vertebral region with at least one screw, rod, plate, or a combination thereof.

According to another aspect, a method for delivering at least one flowable biologic component to a spinal region is provided. The method includes the step of providing the implant as provided herein, and applying the implant to, into, or on a vertebral region of the spine. According to one embodiment, the method further includes the step of securing the implant to the vertebral region with at least one screw, rod, plate, or a combination thereof. According to one embodiment, the vertebral region includes one of a cervical vertebrae in the C1, C2, C3-C6, or C7 position. According to one embodiment, the flowable biologic component further includes one or more vitamin, nutrient, inflammatory inhibitor, antibiotic, cytokine, mineral, growth factor hyaluronic acid, cellular attractant, scaffolding reagent, antibiotic, chemotherapeutic agent, antigen, antibody, enzyme, NSAID, or muscle relaxant. According to one embodiment, the structural material supports inter body spacing of the spine between vertebrae. According to one embodiment, the implant connects the vertebrae at the end plates upon introduction.

According to another aspect, a kit for stabilizing the spine is provided. According to one embodiment, the kit includes an implant as provided herein and, optionally, at least one screw, rod, or combination thereof for securing the implant, and instructions for use thereof.

### DETAILED DESCRIPTION OF THE INVENTION

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

As used herein, “birth tissue” encompasses one or more of the components of the placental organ including, but not limited to, the umbilical cord, the umbilical cord blood, the chorionic membrane, the amnion membrane, the amniotic membrane (fully intact without removal of one or more layers), the Wharton's jelly, the amniotic fluid, and other placental gelatins, cells, and extracellular material.

As used herein, “placental tissue components” encompass one or more of the tissue components of the placental organ including, but not limited to, the umbilical cord, the umbilical cord blood, the amnion membrane, the chorionic membrane, the amniotic membrane, the Wharton's jelly and other placental gelatins, cells and extracellular material.

As used herein, the term “effective amount” refers to an amount of a particular composition sufficient to elicit the desired therapeutic effects.

As used herein, the term “arthrodesis surgery” refers to a procedures for linking, fusing or otherwise welding bones together. Such a surgery may function to prevent motion between vertebral bodies, repair a fracture or stabilize a spinal deformity.

As used herein, the term “decompression surgery” refers to laminectomy, laminotomy, foraminotomy, laminaplasty, or other spinal surgery where spinal fusion or stabilization interventions may be utilized.

As used herein, the term “laminectomy” refers to the surgical procedure for removing the entire lamina, a portion of the facet joints, and any thickened ligaments overlying the spinal cord and nerves.

As used herein, the term “laminotomy” refers to the surgical procedure for removing a small portion of the lamina and ligaments, usually on a single side.

As used herein, the term “foraminotomy” refers to the surgical procedure for removal of bone around the neural foramen and can be performed with a laminectomy or laminotomy.

As used herein, the term “laminaplasty” refers to the surgical procedure for the expansion of the spinal canal by cutting the laminae on one side and swinging the laminae open.

As used herein, the term “C1” and “first cervical vertebra” refer to the atlas or superior-most vertebra in the spinal column which supports the skull, spinal cord and vertebral arteries.

As used herein, the term “C2” and “second cervical vertebra” refer to the axis vertebra or the epistropheus that is the second-uppermost of the vertebrae making up the backbone and allows the head to rotate from its support atop the C1 vertebra where the skull attaches to the neck.

As used herein, the term “C3-C6” and refer to the third to the sixth vertebra sitting just below the C2 axis vertebra and above the C7 vertebrae that are grouped together

As used herein, the term “C7” and “seventh cervical vertebra” refer to the largest and most inferior vertebra in the neck region. The C7 protrudes posteriorly toward the skin at the back of the neck.

As used herein, the term “time release” refers to the delivery of a flowable biologic component gradually over a period of time.

As used herein, the term “biologic” refers to a type of liquid or flowable component that aids in the healing cascade, aids in the spinal fusion process, or a combination thereof.

Provided herein are spinal implants that optionally fuse to the spine upon placement. According to one embodiment, a spinal implant as provided herein stabilizes the spine. According to one embodiment, the spinal implant is compressible and expandable. According to one embodiment, the spinal implant as provided herein delivers one or more biologic components to an area in need of treatment.

According to one embodiment, the spinal implant may aid in preventing spinal stenosis recurrence and eliminating pain associated with an unstable spine or degenerative discs in the spine. The spinal implants as provided herein are particularly useful for stabilization of the vertebrae after a spine decompression surgery. The spinal implant may be include a biologic component that is liquid or flowable. The flowable biologic may be included in, on, or around a structural spinal material. By providing such a biologic component, adhesion, nerve damage, pain, and implant migration are reduced or eliminated. Further, the incidence of implant rejection is substantially reduced thereby minimizing the potential need for additional surgery. Also provided herein are methods of stabilizing and treating the spinal spine with a spinal implant that includes a flowable biologic component.

The spinal implants as provided herein may also act as intervertebral body spacers or additional intervertebral spacer components. According to such an embodiment, the spacers may be utilized to support an inter body space of the spine while maximizing the fusion of the connected vertebral bodies.

According to one embodiment, the structural material may act as a medical delivery vector to deliver or elute a flowable or liquid biologic component to an area in need of treatment. According to one embodiment, the spinal implant may undergo the action of expansion creating a spacial vacuum allowing the implant to wick up and hold at least one flowable biologic component. The spinal implant may hold and deliver the flowable biologic components upon placement in, on or around a target area. According to one embodiment, the vertebral bodies are connected together immediately upon hydration or continued hydration of the structural spinal implant. According to one embodiment, the implant may expand after placement. According to one embodiment, the biologic component may also come in immediate contact with any prepped spinal or vertebral end plates. In the absence of using such an implant as provided herein, connection or fusion and stabilization can typically take an additional 5-10 days.

According to one embodiment, a flowable biologic component is wicked into and delivered to the intervertebral space. The flowable biologic component may be formulated as a resorbable adhesion barrier alloimplant that is applied to the outer surface of the implant. According to an alternative embodiment, the flowable biologic component may be formulated as an injectable formulation or a flowable formulation that is introduced directly around, onto, or into the structural material. The flowable biologic component as provided herein includes one or more vitamins, nutrients, inflammatory inhibitors, antibiotics, cytokines, minerals, growth factors, hyaluronic acids, cellular attractants, scaffolding reagents, antibiotics, chemotherapeutic agents, antigens, antibodies, enzymes, NSAIDs, muscle relaxants, Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a birth tissue composition, or any combination thereof.

A spinal implant as provided herein may be of various shapes and sizes depending on the ultimate use of the implant. According to one embodiment, the spinal implant as provided herein is of a shape and size to function as a structural support for the spine or as a disc or bone that was removed (disc replacement). According to one embodiment, the spinal implant as provided herein is of a shape and size to function as an onlay that includes a plurality or mass of bone fragments that grow together to stabilize the spine and bridge any joint. According to one embodiment, the spinal implant as provided herein is of a shape and size to provide a scaffold or foundation for new bone growth. According to one embodiment, the spinal implant as provided herein is of a shape and size to be placed between an upper and lower vertebral end plate resulting in fusion and stabilization of the spinal point of placement.

According to an one embodiment (not shown), a rod (or plate) is used to aid in prevention of movement. According to one embodiment, prevention of movement allows the spinal bone implant to attach or fuse. According to such an embodiment, screws are placed above and below any bone, such as vertebrae, fused or stabilized by the implant. The screws and rods can be optionally removed at a later time.

The spinal implants as provided here may be made of one or more materials suitable for implantation into the spine of a mammalian patient such as, for example, a human. Materials may be biocompatible with a mammalian patient and/or may have one or more surface coatings or treatments that enhance biocompatible and also reduce or prevent adhesion, nerve damage, pain, and implant migration. Such implant materials may include one or more materials having sufficient bad capability and/or strength to maintain the desired spacing between vertebrae and provide the desired stability.

According to one embodiment, the spinal implants as provided herein may optionally include one or more of a structural material such as, for example, metal, plastic, cancellous bone, demineralized cancellous bone, alloimplant (fresh or fresh-frozen), freeze dried bone alloimplant, demineralized freeze dried bone alloimplant, cortical cancellous bone, or a combination thereof. According to one embodiment, any donors of bone are subject to proper screening. According to one embodiment, the implants as provided herein may optionally include demineralized bone matrix (DBM) that has undergone a process whereby the mineral content is removed. According to one embodiment, demineralized bone matrix functions as a bone implant extender. According to one embodiment, demineralized bone matrix may be mixed with autoimplant bone or other bone provided herein. According to one embodiment, the autoimplant includes bone cells, proteins, and calcified matrix. The autoimplant bone may be harvested from any appropriate portion of a donor's body including, but not limited, a donor's iliac crests, rib or spine. According to one embodiment, demineralized bone matrix may be mixed with alloimplant bone or other bone provided herein. Any alloimplant donor bone material may be obtained from a tissue bank. According to one embodiment, the alloimplant donor bone materials is prepared for use by freezing or freeze-drying to limit rejection.

According to one embodiment, the spinal implants as provided herein may optionally include one or more natural or recombinant bone morphogenetic proteins (BMPs) that are used to stimulate new bone growth. According to a particular embodiment, the bone morphogenetic protein is BMP-2 and BMP-7. According to one embodiment, the spinal implants as provided herein that include one or more bone morphogenetic proteins (BMPs) are suitable for spinal fusion such as an anterior lumbar interbody fusion. According to one embodiment, the spinal implants as provided herein that include one or more bone morphogenetic proteins (BMPs) are suitable for anterior spinal fusions.

According to one embodiment, the spinal implants as provided herein may optionally include one or more synthetic-based bone implant extenders such as, for example, calcium phosphate, tricalcium phosphate, phosphate, calcium sulfate, and bioactive glass. According to one embodiment, the synthetic-based bone implant extender is osteoconductive and biodegradable. The one or more synthetic-based bone implant extenders aid in fusion without a risk for disease transfer. According to one embodiment, the spinal implants as provided herein may optionally include bone marrow aspirate. According to one embodiment, the implants as provided herein may optionally include growth factors, such as platelet derived growth factor and TGF-β to enhance bone healing by promoting mesenchymal stem cell and osteoblast proliferation.

According to a particular embodiment, the spinal implants as provided herein are made from demineralized cancellous bone as set forth in U.S. Pub. No. 20120259425, the content of which is incorporated herein by reference in its entirety. According to one embodiment, the donated bone material is harvested, tested and sterilized by an accredited tissue bank.

According to one embodiment, the spinal implants as provided herein may optionally include one or more bioactive agents. Suitable bioactive agents include, but are not limited to, antimicrobials, antibiotics, antimyobacterial, antifungals, antivirals, antineoplastic agents, antitumor agents, agents affecting the immune response, blood calcium regulators, agents useful in glucose regulation, anticoagulants, antithrombotics, antihyperlipidemic agents, cardiac drugs, thyromimetic and antithyroid drugs, adrenergics, antihypertensive agents, cholnergics, anticholinergics, antispasmodics, antiulcer agents, skeletal and smooth muscle relaxants, prostaglandins, general inhibitors of the allergic response, antihistamines, local anesthetics, analgesics, narcotic antagonists, antitussives, sedative-hypnotic agents, anticonvulsants, antipsychotics, anti-anxiety agents, antidepressant agents, anorexigenics, non-steroidal anti-inflammatory agents, steroidal anti-inflammatory agents, antioxidants, vaso-active agents, bone-active agents, osteogenic factors, antiarthritics, diagnostic agents and progenitor cells, or any combination thereof.

According to one embodiment, the spinal implants as provided herein include one or more synthetic polymers. According to one embodiment, the synthetic polymer is a polycaprolactone, collagen and open porosity polylactic acid polymer. According to one embodiment, the synthetic polymer is a hydrated polymer or hydrogel.

According to one embodiment, the implants as provided herein are compressible and, thus, the implants can be flattened or narrowed under pressure and preserved in the compressed state. According to one embodiment, the implants as provided herein are expandable and, thus, the implants can increase in size, volume or otherwise become enlarged. According to one embodiment, the implants as provided herein are compressible and expandable as result of introduction of at least one biologic component to one or more of the implant components. According to one embodiment, at least one biologic component is introduced to the structural implant material to expand the structural implant material and returned it to its natural state before compression.

According to a particular embodiment, the birth tissue composition as provided herein includes one or more placental tissue components. According to one embodiment, placental tissue components and amniotic fluid must first be obtained from a seronegative, healthy mammal. Potential birth tissue donors providing informed consent are pre-screened during an examination of pre-natal medical records and blood test results. A comprehensive medical history and behavior risk assessment is obtained from the donor prior to donation incorporating U.S. Public Health Service guidelines when the mammal is human. Discussions with the physician(s) or veterinarian and/or the donor mother or donor owner are conducted to identify circumstances that may lead to the exclusion of the donor or donated tissue. Additionally, a physical exam is performed on the donor to determine whether there is evidence of high risk behavior or infection and to determine the overall general health of the donor. The donor may be any mammal that produces birth tissue capable of being processed to form a biologic component as provided herein. Exemplary mammals include pigs, horses, cows, and goats. Thus, the spinal implants, when used in humans, may include xenograft birth tissue.

Infectious disease testing of donor blood specimens is performed for each tissue donor on a specimen collected at the time of donation or within seven days prior to or after donation. Advantageously, the methods that are used to screen for a communicable disease follow the regulations as set forth by the Federal Drug Administration and the American Association of Tissue Banks. Exemplary infectious disease testing includes, but is not limited to, antibodies to the human immunodeficiency virus, type 1 and type 2 (anti-HIV-1 and anti-HIV-2); nucleic acid test (NAT) for HIV-1; hepatitis B surface antigen (HBsAg); total antibodies to hepatitis B core antigen (anti-HBc—total, meaning IgG and IgM); antibodies to the hepatitis C virus (anti-HCV); NAT for HCV; antibodies to human T-lymphotropic virus type I and type II (anti-HTLV-I and anti-HTLV-II); and syphilis (a non-treponemal or treponemal-specific assay may be performed).

Mammalian birth tissue is preferably recovered from a full-term Cesarean delivery of a newborn. Alternatively, human birth tissue is recovered from a full-term vaginal delivery of a newborn. The subsequent steps of preparing the human birth tissue material are performed in a controlled environment (i.e., certified biological safety cabinet, hood or clean room). Instruments, solutions, and supplies coming into contact with the birth tissue material during processing are sterile. All surfaces coming in contact with the birth tissue material intended for transplant are either sterile or draped using aseptic technique.

Once recovered, one or more of the placental tissue components can be removed via a sterile saline solution rinse, blunt dissection, scalpel, or a combination thereof, if necessary. According to one embodiment, the umbilical cord, chorionic membrane, and other gelatins, fluids, cells and extracellular matrix are removed and discarded, leaving the amniotic membrane for further processing. Preferably, the birth tissue material is subject to preparation no more than four hours after recovery to preserve cell viability.

The retained placental tissue components can be placed in a sterile transport solution after aseptic recovery. The sterile transport solution is used to provide an advantageous medium to the natural function of the placental tissue components prior to processing. For example, calcium-rich water can be used as the sterile transport solution to provide a medium to drive undifferentiated cells to become osteogenic when implanted. Throughout the preparation of the birth tissue material, various methods can be used to drive undifferentiated cells to differentiate into specialized cell types including, but not limited to, transport solutions, soaks, particular temperature ranges, and hyperbaric pressure.

The sterile transport solution may include sodium chloride (NaCl) in a concentration range from typically about 0.1% to typically about 35% by weight. The sterile transport solution can also include one or more of Minimum Essential Medium, Dulbecco's Modified Eagle's Medium, Plasma Lyte-A, human albumin 25% solution, calcium-rich water, alkaline ionized water, or acidic ionized water. After delivery to the processing facility, the weight of the placental tissue components can be determined. Thereafter, the placental tissue components can be transferred aseptically to a sterile dish containing Plasma Lyte-A and stored in a quarantine refrigerator pending further processing. The placental tissue components can be removed from the Plasma Lyte-A and cryopreserved according to methods commonly used in the art. According to one embodiment, the cryopreserved components may then be morselized and formulated into an injectable form and/or a flowable material.

The birth tissue material compositions as described herein can be optionally mixed with or administered in combination with bioactive agents such as inflammatory inhibitors, antibiotics, cytokines, minerals, growth factors (e.g., fibrin and/or thrombin), wound healing agents, hyaluronic acid, cellular attractant and scaffolding reagents (e.g., fibronectin) antibiotics, chemotherapeutic agents, antigens, antibodies, enzymes, NSAIDs, muscle relaxants, vectors for gene delivery and hormones.

The spinal implants as provided herein may be used in a variety of surgeries that require bone implants such as, for example, decompression, fusion and disc replacement procedures. Specific procedures include anterior spinal decompression and fusion, spinal discectomy alone without fusion, and anterior spinal foramenotomy without complete discectomy. According to one embodiment, the implants as provided herein may be used in arthrodesis surgery for linking, fusing or otherwise welding spinal bones together. Such a surgery may function to prevent motion between vertebral bodies, repair a fracture or stabilize a spinal deformity.

The implants as provided herein may be used in decompression surgery in the spinal region. Decompression surgery and subsequent placement of the compressible and expandable implants as provided here are typically performed by an orthopedic surgeon.

Surgery is initiated by making a skin incision down the middle of the neck over the appropriate C1-C7 vertebrae. The length of the incision depends on the number of vertebrae that will be subject to the decompression surgery procedure chosen. The neck muscles are then split and moved to either side of each lamina exposing the target spinal vertebra area. During a disc replacement (e.g., total disc arthroplasty), a disc is completely removed and replaced with a spinal implant as provided herein such that the implant is placed between vertebral plates. According to one embodiment, the C1 disc is replaced. According to another embodiment, any one or more of the C3-C6 discs is replaced. According to one embodiment, the C7 disc is replaced. Such a disc replacement results in restoration of normal height to the spine to allow proper area and spacing for nerves. Disc replacement also aids in neck stability while preserving range of motion. Optionally, the surgeon may then retract the dural sac and nerve root to remove any bone spurs or thickened ligaments. The facet joints may then be undercut or trimmed. The joint sparing spinal fusion to stabilize the spine may then be performed.

According to one embodiment, anterior surgery is performed utilizing one or more of the spinal implants as provided herein. Anterior surgery restores spinal lordosis, stabilizes the spine, and decompresses the nerve roots. According to one embodiment, a plane is opened between the carotid artery and the esophagus leading to the anterior aspect of the spine. According to one embodiment, the spinal disc is then removed in its entirety along with any osteophytes at the posterior aspect of the vertebral body. The evacuated disc is then replaced with an implant as provided herein.

According to one embodiment, the spinal implants as provided herein may be used to wick in or up and hold one or more flowable biologic components useful in the healing cascade such that, upon placement, the spinal implants deliver, release or elute such one or more biologic components at the area of placement in the spine making the implant a medical delivery vector. According to one embodiment, the spinal implant slowly delivers, releases or elutes such one or more biologic components over time. Such biologic components useful in the healing cascade include one or more vitamins (calcium, magnesium, vitamin D3, vitamin K, vitamin C, collagen, glucosamine, chondroitin, vitamin B12, iron, copper, zinc, boron, manganese), nutrients, inflammatory inhibitors, antibiotics, cytokines, minerals, growth factors (e.g., fibrin and/or thrombin), wound healing agents, hyaluronic acid, cellular attractant and scaffolding reagents (e.g., fibronectin) antibiotics, chemotherapeutic agents, antigens, antibodies, enzymes, NSAIDs, muscle relaxants, Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a birth tissue composition, or any combination thereof.

A method of delivering at least one flowable biologic component to a spinal region is provided. The method includes the step of introducing at least one flowable biologic component to at least one structural implant material such as, for example, metal, plastic, cancellous bone, demineralized cancellous bone, fresh alloimplant, frozen alloimplant freeze dried bone alloimplant, demineralized freeze dried bone alloimplant, cortical cancellous bone, or a combination thereof to form an compressible and expandable implant. The method further includes the step of introducing the implant to the spine's cervical region or any region of the spine of a patient in need of treatment. According to one embodiment, the at least one flowable biologic component is delivered in a time release manner. According to one embodiment, the flowable biologic component is delivered over a period of time from about 1 minutes to about 1 week. According to such an embodiment, the resulting implant acts as a vector for delivery of at least one flowable biologic component. According to one embodiment, the at least one flowable biologic component includes, for example, one or more vitamins (calcium, magnesium, vitamin D3, vitamin K, vitamin C, collagen, glucosamine, chondroitin, vitamin B12, iron, copper, zinc, boron, manganese), nutrients, inflammatory inhibitors, antibiotics, cytokines, minerals, growth factors (e.g., fibrin and/or thrombin), wound healing agents, hyaluronic acid, cellular attractant and scaffolding reagents (e.g., fibronectin) antibiotics, chemotherapeutic agents, antigens, antibodies, enzymes, NSAIDs, muscle relaxants, Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a flowable birth tissue material composition, or any combination thereof. According to one embodiment, the biologic component aids in fusing the a targeted spinal region. According to one embodiment, the biologic component aids in the healing cascade.

A kit is also provided that includes one or more implants as provided herein. The kit may include various sizes of spinal implants depending on the location of where the implant may be utilized in the spine. The kit may further include tools or other devices useful in selecting, inserting, positioning, and/or securing one or more implants. Tools and devices may include, for example, one or more pins, screws, rods, plates, wires, cables, straps, surgical rope, sutures, or other devices typically used for positioning and securing the implants. The kit further includes at least one set of instructions.

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

We claim:

1. A spinal implant comprising
at least one structural material selected from the group consisting of metal, plastic, cancellous bone, demineralized cancellous bone, fresh alloimplant, frozen alloimplant freeze dried bone alloimplant, demineralized freeze dried bone alloimplant, cortical cancellous bone, or a combination thereof; and
an effective amount of at least one flowable biologic component selected from the group consisting of Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a birth tissue material composition, and any combination thereof,
wherein the effective amount of the flowable biologic component is deposited on, in, or around the structural material such that the structural material wicks or absorbs the flowable biologic component for later delivery of the at least one flowable biologic component upon placement in or on a patient.

2. The implant of claim 1, wherein the birth tissue material composition comprises one or more components of the placental organ from a mammal, the one or more of the components of the placental organ selected from the group consisting of umbilical cord, umbilical cord blood, chorionic membrane, amnion membrane, amniotic membrane, Wharton's jelly, amniotic fluid, and extracellular material.

3. The implant of claim 1, further comprising one or more natural or recombinant bone morphogenetic proteins (BMPs) selected from the group consisting of BMP-2, BMP-7 and a combination thereof.

4. The implant of claim 1, further comprising one or more synthetic-based bone implant extenders.

5. The implant of claim 4, wherein the one or more synthetic-based bone implant extenders is calcium phosphate, tricalcium phosphate, phosphate, calcium sulfate, bioactive glass, or a combination thereof.

6. The implant of claim 1, further comprising one or more bioactive agents. The implant of claim 1, further comprising one or more synthetic polymers.

8. The implant of claim 1, wherein the implant is shaped to fuse to one or more of the C1, C2, C3-C6 or C7 discs.

9. The implant of claim 1, wherein the flowable biologic component further includes one or more vitamins, nutrients, inflammatory inhibitors, antibiotics, cytokines, minerals, growth factors, hyaluronic acids, cellular attractants, scaffolding reagents, antibiotics, chemotherapeutic agents, antigens, antibodies, enzymes, NSAIDs, muscle relaxants,

10. A method of stabilizing a vertebral region of a spine comprising providing the implant of claim 1,
applying the implant to, into, or on a vertebral region of the spine.

11. The method of claim 10, further comprising the step of securing the implant to the vertebral region with at least one screw, rod, plate, or a combination thereof.

12. The method of claim 11, wherein the vertebral region includes one of a cervical vertebrae in the C1, C2, C3-C6, or C7 position.

13. The method of claim 10, wherein the flowable biologic component further includes one or more vitamin, nutrient, inflammatory inhibitor, antibiotic, cytokine, mineral, growth factor hyaluronic acid, cellular attractant, scaffolding reagent, antibiotic, chemotherapeutic agent, antigen, antibody, enzyme, NSAID, or muscle relaxant.

14. The method of claim 10, wherein the structural material supports inter body spacing of the spine between vertebrae.

15. The method of claim 14, wherein the implant connects the vertebrae at the end plates upon introduction.

16. A method for delivering at least one flowable biologic component to a cervical spinal region comprising
forming an implant by introducing at least one flowable biologic component to at least one structural implant material selected from the group consisting of metal, plastic cancellous bone, demineralized cancellous bone, fresh alloimplant, frozen alloimplant freeze dried bone alloimplant, demineralized freeze dried bone alloimplant, cortical cancellous bone, and a combination thereof; and
introducing the implant to the cervical region of a patient in need treatment,
wherein at least one flowable biologic component is delivered to the cervical spinal region.

17. The method of claim 16, wherein the at least one flowable biologic component is a biologic selected from the group consisting of Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, platelet rich plasma, a flowable birth tissue material composition, and a combination thereof.

18. A kit for stabilizing or treating a spine comprising
an implant as provided in claim 1;
optionally, at least one screw, rod, or combination thereof for securing the implant to the spine; and
instructions for use thereof.
